(19) United States
(12) Patent Application Publication
(10) Pub. No.: US 2018/0280450 A1
(43) Pub. Date: Oct. 4, 2018

(54) METHOD OF PROMOTING IMMUNE HEALTH USING THE WATER-SOLUBLE COMPONENT FROM GENUS EUGLENA ORGANISM

(71) Applicant: KEMIN INDUSTRIES, INC., Des Moines, IA (US)

(72) Inventors: Robert Bernard LEVINE, Ann Arbor, MI (US); Geoffrey Paul HORST, Grosse Pointe Farms, MI (US); John A. MINATELLI, Mount Dora, FL (US); W. Stephen HILL, Ocala, FL (US); Patricia M. O'CONNELL, Apopka, FL (US)

(21) Appl. No.: 15/939,050

(22) Filed: Mar. 28, 2018

Related U.S. Application Data
(60) Provisional application No. 62/477,594, filed on Mar. 28, 2017.

(51) Int. Cl. A61K 35/68; A61K 45/06; A23L 33/105; A23L 33/135

(52) U.S. Cl. CPC A61K 35/68; A61K 45/06; A23V 2200/324; A23L 33/135; A23V 2002/00; A23L 33/105

(57) ABSTRACT

The present invention relates to compositions and methods for promoting the immune health of an animal or human by administering compositions or mixtures of water-soluble components derived from a genus Euglena organism that are capable of stimulating immune system activity in the absence of beta-glucan.

### CROSS REFERENCE TO RELATED APPLICATIONS

This application claims the benefit of and priority to U.S. Provisional Application No. 62/477,594, filed Mar. 28, 2017, entitled “METHOD OF PROMOTING IMMUNE HEALTH USING THE WATER-SOLUBLE COMPONENT FROM GENUS EUGLENA ORGANISM,” the entirety of which is incorporated herein by reference.

### FIELD OF THE INVENTION

The present invention relates to compositions or mixtures derived from a genus Euglena organism, such as Euglena gracilis algae, for promoting immune health in humans and animals. Another aspect of the invention relates to methods for administering compositions and mixtures derived from Euglena gracilis algae that are capable of stimulating immune system activity in the absence of beta glucan.

### BACKGROUND OF THE INVENTION

Beta-glucans have been used for years as dietary supplements to stimulate immune system activity and promote immune health in humans and animals. They have been the subject of numerous clinical trials. Beta-glucans are D-glucose polysaccharides having a linear backbone connected by beta-glycosidic bonds. Beta-glucans are found in different organisms, including yeast, mushrooms, fungi, bacteria, cereal grains, algae and other organisms. Yeast is a common source of beta-glucan and when derived from yeast, the beta-glucan is extracted from the yeast cell wall using different processes, including solvent extraction. Other beta-glucans can be derived from mushrooms, oats, barley and kelp and also extracted from the cell wall such as by solvent extraction. These beta-glucans are typically very expensive because the beta-glucans must be carefully extracted from the cell wall and the initial beta-glucan content in the raw material being extracted is low.

The beta-glucan structure is complex and different variations occur with different molecular weights and branching structures as a result of the use of different source organisms, production methods, and extraction techniques, which all impact the efficacy and suitability of a particular beta-glucan. Most commercial beta-glucan products are manufactured from yeast-derived beta-1,3/1,6-glucans or oat derived beta-1,3/1,4-glucans, which all have been found useful for reducing cholesterol and promoting immune health and stimulating immune system activity.

Humans do not synthesize beta-glucans and the human immune system recognizes the beta-glucan compounds as a foreign substance. The body's innate immune system responds via pattern recognition receptors (PRR) that are expressed by immune and other cells. These receptors recognize microbe-associated molecular patterns (MAMPs). The beta-glucans can be considered a major MAMP for PRR-mediated sensing. It has been determined that an important PRR for beta-glucan is the Dectin-1 receptor, the Complement Receptor 3 (CR3) and Toll-Like Receptors (TLR) found on different immune cells such as the monocytes, macrophages, dendritic cells, neutrophils, eosinophils, and natural killer cells. A cascade of innate and adaptive immune responses such as phagocytosis, oxidative bursts, and the production of cytokines, chemokines, dendritic cells and macrophages are induced when the beta-glucans bind to the Dectin-1. Of course, the route of administration for a beta-glucan also affects the immune function and how the body responds. For this reason, the size and branching of the beta-glucan becomes important. Also important are any other components associated with the beta-glucan, including different insoluble and soluble components that may be added or are inherent to the source.

Different organisms produce different beta-glucans and they are not all equally effective. Some algae or protist-derived sources have been found to have greater beta-glucan content than those sources where the beta-glucan is extracted from the cell wall. Beta-glucan produced from an algae or protist such as Euglena gracilis algae is typically linear with fewer side branches and may include other water-soluble and insoluble components and unique compounds that aid in promoting immune function and health. This may be due to a similar evolutionary history that algae and protists have when compared to the development of fungi, plants or bacteria that provide other sources of beta-glucan. Also, the production of beta-glucan from an algae or protist meal does not require expensive solvent-based extraction processes since the beta-glucan is not extracted from the cell walls, but instead is extracted from the cell itself, which stores the beta-glucan typically in a form known as paramylon. The genus Euglena organism such as Euglena gracilis accumulates beta-glucan as water-insoluble granules in its cytoplasm and uses this form of beta-glucan as a carbohydrate energy storage molecule.

An example of the genus Euglena organism often used to produce beta-glucans is Euglena gracilis algae. An example of beta-glucan derived from Euglena gracilis algae is disclosed in U.S. Pat. No. 9,574,217 and U.S. Patent Publication No. 2013/0216586, the disclosures which are hereby incorporated by reference in their entirety. In these references, the beta-glucan is derived from the Euglena gracilis algae, which is heterotrophically grown and is golden in color. The beta glucan consists essentially of an unbranched beta-1,3-glucan having an average molecular weight of about 200 to 500 kDa, and is typically in the native form of paramylon as a water-insoluble granule. The paramylon is a linear polymer and lacks most of the beta(1,6), beta(1,4), and beta(1,2) bonds and side branching structures of the beta-glucans from yeast and similar sources. The genus Euglena organism produces almost exclusively beta-1,3-glucan with few side branches as compared to yeast beta-glucans that contain a beta-1,3-glucan backbone that is substituted with beta(1,6) side chains that are 2 to 3 glucose units long every 10 to 30 glucose units.

The beta-glucan disclosed in the incorporated by reference '217 patent and '586 patent publication includes a triple-helix structure of linear beta-1,3-glucan that is stabilized by different types of hydrogen bonding, including intermolecular hydrogen bonding formed between different chains in the same x-y plane, intramolecular hydrogen bonding formed between adjacent oxygen atoms in the same chain, and intramolecular hydrogen bonding formed between different chains in a different x-y plane. This triple helix structure is stable over different temperatures. The polymer is water insoluble and typically formed as high purity paramylon. Typically, the beta-1,3-glucan particle derived from the Euglena gracilis algae is between about 0.2 and 5.0 microns in diameter. It is an insoluble product and has a molecular weight of about 200 to 500 kDa.

The incorporated by reference '586 application and '217 patent disclose techniques for growing Euglena gracilis algae heterotrophically and isolating the pure paramylon as a high purity beta-1,3-glucan that may test greater than 98% beta-glucan with about 2% moisture. In one example, it is formed as a white powder with little taste or odor. Another beta-glucan product produced from Euglena gracilis algae is a dry yellow powder that is clean and free of allergens and contains about 50% beta-1,3-glucan as a powdered whole cell Euglena.

### SUMMARY OF THE INVENTION

This summary is provided to introduce a selection of concepts that are further described below in the detailed description. This summary is not intended to identify key or essential features of the claimed subject matter, nor is it intended to be used as an aid in limiting the scope of the claimed subject matter.

One aspect of the present invention relates to a method of promoting the immune health of a human or animal or stimulating immune system activity including administering to a human or animal a composition or preparation derived from a genus Euglena organism, for instance a water-soluble component or fraction where the beta-glucan is separated from the mixture. According to one embodiment of the present invention, the mixture is obtained through a filtration process. The mixture may be obtained through centrifugation or other known separation processes. The genus Euglena organism may be derived from a heterotrophically, phototrophically or mixotrophically grown genus Euglena organism. The separation step may include filtration, including for instance sterile filtration, and may comprise filtering an aqueous mixture of milled whole cell genus Euglena organism through a filter, by way of example 1 micron or less, or specifically 0.2 micron or less, filter. The aqueous mixture of the milled whole cell genus Euglena organism may be made by milling dried whole cell genus Euglena organism to a fine powder and adding sterile water to form an aqueous mixture of the milled whole cell genus Euglena organism. The aqueous mixture of milled whole cell genus Euglena organism may be bead milled and centrifuged before sterile filtering. The water-soluble component derived from the genus Euglena organism may be administered orally, topically by ointment or cream, injected using a nasal spray, subcutaneously, transdermally, intradermally, intravenously or gastrointestinally. The genus Euglena organism in a preferred aspect may be a Euglena gracilis algae.

Another aspect of the present invention relates to a method for promoting the immune health of an animal or human or stimulating immune system activity includes administering a whole-cell preparation from the genus Euglena including water-soluble cytoplasm components wherein the water-soluble component is capable of stimulating immune system activity in the absence of beta-glucan. The Euglena may be fermented heterotrophically, mixotrophically, and phototrophically. The whole-cell preparation may be Euglena gracilis and may be delivered as a human dietary supplement in a suspension, capsule, or tablet form. The whole-cell preparation may be delivered as an ingredient in food. The animal may include but is not limited to cattle, swine, poultry or a companion animal.

A composition may include the combination with other innate or adaptive immune modulated compounds such as a probiotic, an inactivated bacteria, a beta-1,3:1,6-glucan, a beta-1,3-glucan, an algae extract, or a carotenoid. In at least one embodiment, the probiotic may be a Bacillus or Lactobacillus; the inactivated bacteria may be a Bacillus or Lactobacillus and the beta-glucan may be derived from a yeast, mushroom or Euglena; the algae extract may be derived from Arthrospira or Haematococcus; and the carotenoid may be astaxanthin.

### BRIEF DESCRIPTION OF THE DRAWINGS

Other objects, features and advantages of the present invention will become apparent from the detailed description of the invention which follows, when considered in light of the accompanying drawings in which:

FIG. 1 is a high-level flowchart illustrating the process of producing the water-soluble component in accordance with a non-limiting example.

FIG. 2 is a graph showing a cell viability MTT assay for the whole-cell preparation, including the water-soluble component as used in the sterile in vitro testing in accordance with a non-limiting example.

FIG. 3 is a graph showing a cell viability MTT assay for the insoluble paramylon test product in accordance with a non-limiting example.

FIG. 4 is a bar chart showing the CD69 expression on NK cells for the water-soluble component as the test product in accordance with a non-limiting example.

FIG. 5 is a graph showing the CD69 expression on NK cells for the insoluble paramylon test product in accordance with a non-limiting example.

FIG. 6 is a bar chart for the CD25 expression on NK cells for the water-soluble component test product in accordance with a non-limiting example.

FIG. 7 as a bar chart for the CD25 expression on NK cells for the insoluble paramylon test product in accordance with a non-limiting example.

FIG. 8 is a bar chart for the CD69 expression on NKT cells for the water-soluble component test product in accordance with a non-limiting example.

FIG. 9 is a bar chart for the CD69 expression on NKT cells for the insoluble paramylon test product in accordance with a non-limiting example.

FIG. 10 is a bar chart for the CD25 expression on NKT cells for the water-soluble component test product in accordance with a non-limiting example.

FIG. 11 is a bar chart for the CD25 expression on NKT cells for the insoluble paramylon test product in accordance with a non-limiting example.

FIG. 12 is a bar chart for the CD69 expression on T-cells for the water-soluble component test product in accordance with a non-limiting example.

FIG. 13 is a bar chart for the CD69 expression on T-cells for the insoluble paramylon test product in accordance with a non-limiting example.

FIG. 14 is a bar chart for the CD25 expression on T-cells for the water insoluble component test product in accordance with a non-limiting example.

FIG. 15 is a bar chart for the CD25 expression on T-cells for the insoluble paramylon test product in accordance with a non-limiting example.

FIG. 16 is a bar chart for the CD69/CD25 expression on lymphocytes for the water-soluble component test product in accordance with a non-limiting example.

FIG. 17 is a bar chart for the CD69/CD25 expression on lymphocytes for the insoluble paramylon test product in accordance with a non-limiting example.

FIG. 18 is a bar chart for the CD69 expression on non-T non-NK lymphocytes for the water-soluble component test product in accordance with a non-limiting example.

FIG. 19 is a bar chart for the CD69 expression on non-T non-NK lymphocytes for the insoluble paramylon test product in accordance with a non-limiting example.

FIG. 20 is a bar chart for the CD25 expression on non-T non-NK lymphocytes for the water-soluble component test product in accordance with a non-limiting example.

FIG. 21 is a bar chart for the CD25 expression on non-T non-NK lymphocytes for the insoluble paramylon test product in accordance with a non-limiting example.

FIG. 22 is a bar chart for the CD69 expression on monocytes for the water-soluble component test product in accordance with a non-limiting example.

FIG. 23 is a bar chart for the CD69 expression on monocytes for the insoluble paramylon test product in accordance with a non-limiting example.

FIG. 24 is a table that compares the eighttest products for immune cell activation.

FIGS. 25 and 26 are tables that compare the eight test products for cytokine effects in accordance with a non-limiting example.

### BACKGROUND OF THE INVENTION

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

It has been found that the water-soluble component derived from a genus Euglena organism, such as the Euglena gracilis algae, which can be obtained by filtering an aqueous mixture of milled whole cell genus Euglena organism, for example, contains a potent water-soluble antigen not related to the insoluble and high molecular weight paramylon also found in the genus Euglena organisms, such as the Euglena gracilis algae. This water-soluble component shows very broad and potent immune cell activation and causes extensive release of immune cross-talk cytokines generated by immune cells coming into direct contact with the water-soluble component and increases the absolute proportion of many immune cell types. This surprising discovery that the water-soluble component has substantial benefits in these areas opens new possibilities for its use and modified composition. This discovery was made when investigating and screening different test products that were validated and compared with each test product's biological activity and potency.

According to at least one embodiment of the present invention, filtering the water-soluble component, for instance using a 0.22 (0.2) micron filter, results in a water-soluble component or mixture that does not contain beta-glucan. The absence of beta-glucan was confirmed microscopically and with NMR data before adding the water-soluble component to the cell cultures. Surprisingly, the cell-based immune activation assays showed immune activating properties even in the absence of beta-glucan. This unexpected result has been repeated and confirmed, and will be explained in greater detail below. Although described herein as a “water-soluble component,” it should be understood that the components or mixtures may be derived or extracted through known techniques, including but not limited to extraction with water, alcohol or other polar solvents.

Bioassay screening of nutraceutical ingredients and one blend were performed to evaluate their potency and mechanisms of action. The screening involved testing in cell cultures involving human immune cells. As part of this process, selected immune ingredients currently on the market were compared for similarities and differences of mechanisms of action, as well as efficacy (active dose comparison). Eight Test Products labeled Test Products A-H, as noted in the various tables and explained below were employed in this study.

As explained in greater detail below, the surprising discovery was made that a water-soluble component derived from a genus Euglena organism such as Euglena gracilis algae and contained in the whole cell and after sterile filtering through a 0.2 micron or less filter an aqueous mixture of heterotrophically grown, milled whole cell genus Euglena organism such as Euglena gracilis algae, which in an example comprises about 50 to 60% beta-1,3-glucan, is a very broad and potent immune cell activator and causes extensive release of immune cross-talk cytokines generated by the immune cells coming into direct contact with the water-soluble component. This water-soluble component has also been found to increase the absolute populations of many immune cell types. In the description below with the various graphs, bar charts and tables, this water-soluble component corresponds to Test Product C. This water-soluble fraction (or component) derived from the genus Euglena organism such as Euglena gracilis algae contains a potent water-soluble antigen not related to the insoluble and high molecular weight paramylon found in the genus Euglena organism and more particularly the Euglenagracilis algae. The high purity and insoluble paramylon in the description below corresponds to Test Product D. This water-soluble component derived from the genus Euglena organism and more specifically the Euglena gracilis algae is also referred to in this description generally as derived from whole cell Euglena gracilis algae and a whole cell Euglena.

This water-soluble component derived from the genus Euglena organism, for instance Euglena gracilis algae, may be administered orally, topically by ointment or cream, injected using a nasal spray, subcutaneously, transdermally, intradermally, intravenously or gastrointestinally. It can be encapsulated in one or more capsules or supplied as patches or other techniques known to those skilled in the art.

For comparison purposes, Test Product C is also referred to as the Euglena gracilis algae corresponding to a source that in an example originally may have had 50-60% beta-1,3,-glucan content in the milled whole cell Euglena gracilis algae and is compared in the following charts and explanations to the paramylon sample as Test Product D that was formed from about 95% pure beta-glucan.

During further processing, various additives and other components may be added as a formulated product and may include surfactants, antioxidants, stabilizers and/or preservatives that may extend the shelf life and preserve any algae derived products and components that may be added, such as various oils, including omega-3 fatty acids, ALA and LA, or EPA and DHA. The oils may include a krill oil, fish oil, marine oil, a seed oil, or other oils that may also act as surfactants to help or aid the water-soluble component to be more bioavailable. Other additives may be added, including vitamins and trace metals. It is possible to include lipases or enzymes.

According to at least one embodiment, a mixture of antioxidants may be added. The selected antioxidants may include a mixture of selected lipophilic and hydrophilic antioxidants. In another example, the composition comprises lipophilic antioxidants either alone or in combination with at least one of: a) phenolic antioxidants including at least one of sage, oregano, and rosemary; b) tocopherol, c) tocotrienol(s), d) carotenoids including at least one of astaxanthin, lutein, and zeaxanthin; e) ascorbylacetate; f) ascorbylpalmitate g) Butylated hydroxytoluene (BHT); h) Docosapentaenoic Acid (BHA) and i) Tertiary Butyl hydroquinone (TBHQ). In another non-limiting example, a hydrophilic antioxidant or sequesterant is added that includes hydrophilic phenolic antioxidants, including at least one of grape seed extract, tea extracts, ascorbic acid, citric acid, tartaric acid, and malic acid.

The composition of matter may include dietary supplement ingredients such as docosahexaenoic acid (DHA) and/or eicosapentaenoic acid (EPA). It may include pectin or gelatin based confectionary dietary supplement delivery systems. EPA, DHA, docosahexaenoic acid (DPA) or gamma-linlolenic acid (GLA), fish oil, krill oil, krill oil concentrate, borage oil, evening primrose oil, olive oil or other plant, animal or algal based seed or fruit oils may be admixed therein either alone or in combination. Lipophilic antioxidants may be added either alone or in combination with at least one of a) phenolic antioxidants including at least one of sage, oregano, and rosemary; b) tocopherol, c) tocotrienol(s), d) carotenoids including at least one of astaxanthin, lutein, and zeaxanthin; e) ascorbylacetate; f) ascorbylpalmitate g) Butylated hydroxytoluene (BHT); h) Docosapentaenoic Acid (BHA) and i) Tertiary Butyl hydroquinone (TBHQ). A hydrophilic antioxidant or sequesterant may include hydrophilic phenolic antioxidants including at least one of grape seed extract, tea extracts, ascorbic acid, citric acid, tartaric acid, and malic acid.

Oils may be used either alone or advantageously in combination with other ingredients and additives, for example, algae, plant or fish derived alpha-linolenic acid (ALA) or linoleic acid (LA), metabolites such as eicosapentaenoic acid (EPA), docosapentaenoic acid (DPA), gamma-linlolenic acid (GLA) or docosahexaenoic acid (DHA), or any combination thereof. The composition may be incorporated into appropriate foods, beverages or dietary supplements. By way of a non-limiting example, the composition may also be used for the prevention or mitigation of such diseases as cardiovascular disease, arthritis, pain, blood clotting, dry eyes and brain health.

Lipophilic antioxidants may be added to increase the room temperature stability of the resulting oil. The lipophilic antioxidants may be added either alone or in combination with at least one of a) phenolic antioxidants including at least one of sage, oregano, and rosemary; b) tocopherol, c) tocotrienol(s), d) carotenoids including at least one of astaxanthin, lutein, and zeaxanthin; e) ascorbylacetate; f) ascorbylpalmitate g) Butylated hydroxytoluene (BHT); h) Docosapentaenoic Acid (BHA) and i) Tertiary Butyl hydroquinone (TBHQ). The resulting dewatered seed oil can be treated with bleaching clay or activated carbon.

The composition may be treated with a lipophilic or hydrophilic antioxidant(s). The hydrophilic antioxidant or sequesterant can be formed from hydrophilic phenolic antioxidants including at least one of grape seed extract, tea extracts, ascorbic acid, citric acid, tartaric acid, and malic acid.

Antioxidants may be added depending on the composition additives and what is mixed, such as adding the Valensa OTB® per oxidation blocker system as a stabilizer. Stabilization may extend shelf life. The OTB® per oxidation blocker system used by Valensa is 100% natural, non-GMO, and protects any sensitive oils that may be added. The OTB® per oxidation blocker is a synergistic proprietary formulation of powerful natural compounds including astaxanthin, phenolic antioxidants and natural tocopherols. This technology prevents destructive oxidative, photochemical and rancification reactions. It protects expensive and sensitive compounds such as carotenoids and polyunsaturated fatty acids and other additives and may boost the effectiveness of other antioxidants such as vitamin E because it chemically quenches stable vitamin E free radicals. The antioxidants have in vivo activity to protect both products and people.

According to at least one embodiment, probiotics may be added to aid in immune function. As is known, probiotics are microorganisms that are beneficial for the host, and more particularly, the human host. They provide measurable health benefits to the digestive tract and interact with other bacteria in the intestine and other sections of the alimentary canal.

These probiotics promote better digestion and support production of proteases (protein digesting enzymes) and lipases (fat-digesting enzymes), and creation of essential vitamins. In the GI (gastrointestinal) tract, probiotics manufacture several B vitamins and vitamin K. These probiotics also can balance intestinal bacteria after antibiotic therapy. Antibiotics disrupt probiotic populations in the lower GI tract, potentially causing unpleasant side effects during and after therapy. Studies show that the large intestinal ‘ecosystem’ returns to its pre-antibiotic balance more quickly following antibiotic therapy when probiotics are administered.

There is some competitive inhibition of harmful or ‘bad’ bacteria using probiotics. ‘Good’ bacteria (probiotics) compete with ‘bad’ bacteria for fuel and space, making it difficult for harmful bacteria to gain presence in the GI tract. Some probiotics produce natural antibiotic substances (like acidophilin, in the case of L. acidophilus DDS-1) that directly attack harmful bacteria, including some of the most feared food-borne pathogens. Thus, today's growing problem of antibiotic-resistant bacteria makes the need for alternatives all the more urgent.

There is also a balancing of the immune response. Probiotics impact the immune system, stimulating the production of immunoglobulins (antibodies) and cytokines (chemicals made in the body that modulate inflammation).

There is also a direct effect on the genes of some pathogens. Probiotics appear to have the capacity to affect the expression of those genes in ways that reduce pathogens' virulence. (For example, see Corr S C, Hill C, Gahan CG, “Chapter 1: Understanding the Mechanisms by Which Probiotics Inhibit Gastrointestinal Pathogens,” Adv Food Nutr Res 2009; 56:1-15.)

There are major benefits that probiotic products offer: 1) digest foods and alleviate digestive disorders; 2) enhance synthesis of B vitamins and improve absorption of calcium; 3) keep E. coli in check; 4) promote vagina health and keep yeast in check; 5) improve immune function; 6) support the management of acne; and 7) help maintain normal cholesterol.

As persons of ordinary skill will readily appreciate, all probiotic products are not the same. The name Acidophilus or probiotic does not mean anything unless the probiotic product: 1) contains the right strain{s); 2) is viable; 3) remains stable and viable for a long period; 4) has ability to survive in the intestine; and 5) produces beneficial effects in the intestine.

It is possible to use a probiotic, prebiotic, or inactivated bacteria as a dietary ingredient used in functional foods and dietary supplements. This probiotic could be a gram-positive spore-forming rod that is aerobic to microaerophilic in nature. It may be manufactured as a pure cell mass consisting primarily of Bacillus Coagulens. The pure cell mass may be spray-dried with maltodextrin to achieve the desired concentration of 15×10⁹ CFU/G for a finished product. With this product, the spores wait to germinate and grow until they reach the intestines and the protective shell allows it to survive harsh manufacturing processes, product shelf life and the digestive system. It can be used in many different food ingredients and drinks. It may be combined with the prebiotic soluble fiber such as in fructooligosaccharides. It has anti-inflammatory effects. The fructooligosaccharide as the prebiotic is not digested in the body and will increase the amount of material in the intestinal tract and stimulate the elimination process to support regularity.

Another aspect of the present invention relates to a method for promoting the immune health of an animal or human or stimulating immune system activity includes administering a whole-cell preparation from the genus Euglena including water-soluble cytoplasm components. The Euglena may be fermented heterotrophically, mixotrophically, and phototrophically. The whole-cell preparation may be Euglena gracilis and may be delivered as a human dietary supplement in a suspension, capsule, or tablet form. The whole-cell preparation may be delivered as an ingredient in food or animal feed. The animal may include but is not limited to cattle, swine, poultry or a companion animal.

A composition may include or be administered in combination with other innate or adaptive immune modulated compounds such as a probiotic, an inactivated bacteria, a beta-1,3:1,6-glucan, a beta-1,3-glucan, an algae extract, or a carotenoid. The probiotic may be, but is not limited to, a Bacillus or Lactobacillus. The inactivated bacteria may be a Bacillus or Lactobacillus and the beta-glucan may be derived from a yeast, mushroom or Euglena. The algae extract may be derived from Arthrospira or Haematococcus and the carotenoid may be astaxanthin.

### EXAMPLES

There now follows greater details of the identification and preparation of the various Test Products A-H followed by a more detailed comparison and specific test explanation between the water-soluble component derived from the Euglena gracilis algae as Test Product C and the paramylon sample as Test Product D, followed by an overview of the results from testing Test Products A-H.

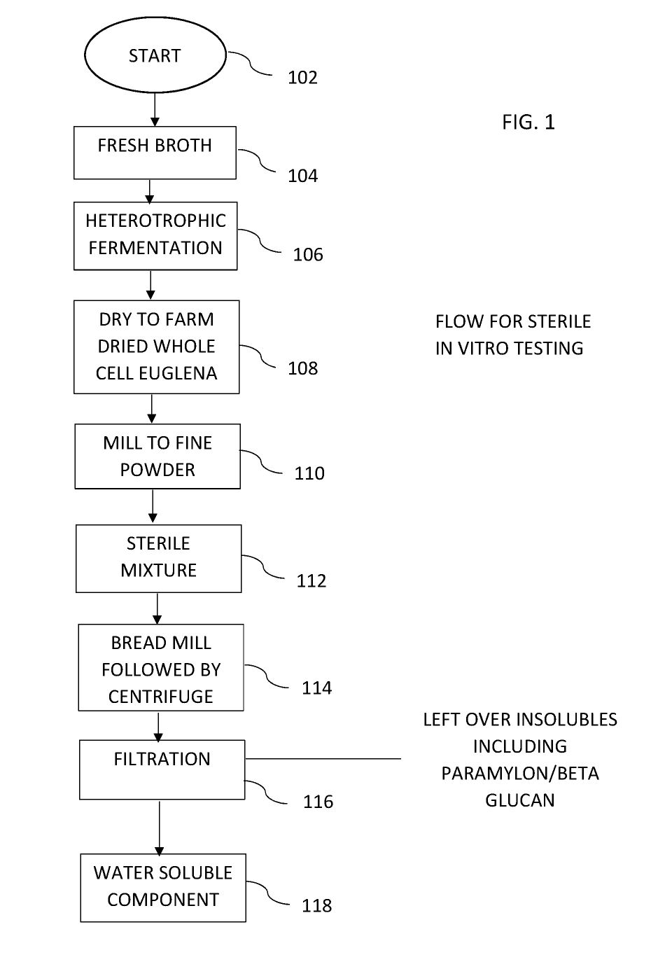

For a general understanding of the Test Product C as the water-soluble component of the invention, a high-level flowchart as an example for producing this water-soluble component is shown in FIG. 1. This water-soluble component is derived from the Euglena gracilis algae as a whole cell algae that is ground and may include in one aspect 50-60% beta-1,3,-glucan. The process 100 starts (Block 102) as a fresh broth of Euglena is prepared (Block 104) and fermented heterotrophically (Block 106) in a preferred aspect. It is then dried (Block 108) to form a whole dried genus Euglena organism as preferred Euglena gracilis algae and then milled to a fine powder (Block 110). Sterile water is added to form an aqueous mixture of milled whole cell genus Euglena organism as the preferred Euglena gracilis algae (Block 112). This aqueous mixture of milled whole cell genus Euglena organism is bead milled and centrifuged (Block 114) before sterile filtering such as through a 0.2 micron filter (Block 116). The cell-free permeate passes through the filter as the filtrate effluent and forms the water-soluble component (Block 118).

As discussed below, Test Products A and B are yeast-based products as branched chain, yeast-based 1,3-1,6 beta-glucans, which are the current standard of care and supplements for immune products apart from Vitamin C and zinc. The analysis of samples of both Test Products A and B prior to the study indicate both samples contained about 70% pure branched beta-glucan and may have had some water-soluble components. However, if such water-soluble components were available in the yeast-based Test Products A or B, they were inactive.

Test Product C is the water-soluble component derived from the genus Euglena organism described above with the abbreviated product name EG-AS and supplied by Algal Scientific Corporation. Test Product D is a pure beta-glucan sample of at least 95% purity (paramylon) sample from Euglena gracilis algae with the abbreviated product name PAR-AS and also supplied by Algal Scientific Corporation at the time, which has since become a part of Kemin Industries, Inc.

Test Product E is an ethanolic extract of Spirulina.

As to the water-soluble component of Test Product C, there are no located reports that any water-soluble component derived from Euglena gracilis algae such as Test Product C was tested and reported to have the activity as now observed. The pure paramylon test Sample D was essentially inactive as a beta-glucan because it was insoluble and did not make it, via the sample preparation method, to the human immune cultured cells. Test Product D for the paramylon is completely insoluble in water. It is evident from the results discussed below in detail that Test Product C for the water-soluble component derived from the whole cell Euglena gracilis algae exhibits very broad immune-stimulating activity. Test Product F is a low molecular weight bioactive peptide complex extracted from the colostral whey fraction. This sample includes an array of peptides and growth factors naturally occurring in colostrum to supportimmune health. Test Product G is a Euglena gracilis lysate and Test Product H is from Valensa International as the Immunum™ Chew containing German brewer's yeast, astaxanthin, New Zealand Manuka honey, and Spirulina extract.

The water-soluble Test Products were prepared by taking 0.25 grams of powder into 5 mL of physiological saline. The suspension was incubated under gentle agitation for one hour, after which any remaining solids were removed by centrifugation. Insoluble, particulate Test Products were prepared by taking 0.5 grams of powder into 3 mL of physiological saline. One (1) mL ceramic beads (200 uM zirconium) were added, and bead milling performed by 10 cycles of repeated 60-second pulsing (vortex high speed, 60 one (1) second pulses) with placement of samples in an ice-bath between cycles. Subsequently, seven (7) mL of physiological saline was then added to bring the samples to a 50 mg/mL final concentration. Microscopy before and after bead milling verified the breakdown of particulate matter in all non-soluble products, however, only to a minor degree in two of the non-soluble products.

The Test Products were prepared for addition to cell cultures by centrifugation at 2400 rpm for 10 minutes followed by sterile filtration, using 0.22 micron cellulose acetate filters, generally referred to as a 0.2 micron filter, which as known to those skilled in the art is the cut-off for generally accepted sterile filtering. For the non-soluble products, it is probable that only finer particles in the ground powders were introduced into cell cultures, while larger particles were removed during the filtration process.

Each Test Product was tested across a very broad concentration dose ranging from 5 mg/mL to 0.000005 mg/mL. This range was selected after reviewing literature for each Test Product, taking into account that in some instances early research publications may have been done on refined extracts produced in a laboratory and possibly may not compare to the same product currently on the market.

In terms of cellular activation, most of the effects were seen in the 0.005 to 5 mg/mL dose range.

It is well known that a cell viability assay is often used as a preparatory step when starting work on the biological effects of complex natural products. The data generated from this testing was used to identify the most promising products for subsequent immune cell testing. The viability testing was performed using a MTT screening assay, which utilizes a dye that changes color dependent on mitochondrial function, which as those skilled in the art know is directly related to cellular metabolic activity and viability. Healthy cells metabolize the MTT dye and turn the cultures purple. When a reduction in color is measured, this is linked to reduced cellular viability, either as a result of direct killing, or inhibition of mitochondrial function leading to cell death. When an increase in color is measured, this has multiple possible explanations: 1) increased cell numbers (growth); 2) increased mitochondrial mass per cell; or 3) increased mitochondrial function (energy production).

Many products have the capacity to activate immune cells and modulate the regulatory responses. Due to the fact that the gut mucosa contains a large volume of immune tissue, consumed products come into contact with immune cells, such as antigen-presenting cells, regulatory cells, and immune active cells. Testing of natural products on immune cells harvested from peripheral blood is a model for some of the potential immune activating and modulating activities that a natural product may trigger upon consumption.

In addition, many products have the capacity to alter inflammatory responses. As knowledge about inflammation continues to increase, it is clear that for a person to be healthy, the person must be able to produce an inflammatory response to a challenge, but must also be capable of resolving this inflammatory response in a timely manner.

Human peripheral blood mononuclear cell (PBMC) cultures were used for this testing. A set of cultures was left untreated as negative control cultures for immune activation. Triplicate sets of cultures were treated with serial dilutions of the test product. The inflammatory bacterial lipopolysaccharide LPS from E. coli was used as a positive control for activation. The cultures were incubated for 24 hours, after which the cells and the culture supernatants were harvested and used to monitor the reactions in each culture. This process is described, for example, in the articles entitled, “Antioxidant and Anti-Inflammatory Properties of an Aqueous Cyanophyta Extract Derived from Arthrospira Platensis: Contribution to Bioactivities by the Non-Phycocyanin Aqueous Fraction,” by Jensen et al., “Antioxidant, Anti-Inflammatory, Anti-Apoptotic, and Skin Regenerative Properties of an Aloe Vera-Based Extract of Nerium Oleander Leaves (nae-8(R)),” by Benson et al., and “West African Sorghum Bicolor Leaf Sheaths Have Anti-Inflammatory and Immune-Modulating Properties In Vitro,” by Benson et al., the disclosure of which are hereby incorporated by reference in their entireties.

The cells were stained with a combination of monoclonal antibodies to monitor activation status, and analyzed by multi-parameter flow cytometry, using an acoustic dual-laser Attune® flow cytometer. The analysis included fluorescent markers for CD3, CD25, CD56 and CD69. This combination allowed monitoring of changes to monocyte/macrophages, as well as activation of natural killer (NK) cells, natural killer T (NKT) cells, and T lymphocytes. The staining with CD3 and CD56 allowed technicians to identify CD3-CD56+ NK cells, CD3+ CD56− T lymphocytes, and CD3+CD56+ NKT cells. The combination also allowed technicians to analyze the CD3−CD56− non-T non-NK lymphocytes for activation markers. For each of these populations, the expression level of the activation marker CD69 was examined, as well as the Interleukin-2 (IL-2) receptor CD25.

The culture supernatants from each culture were used for the testing of a broad panel of pro-inflammatory and anti-inflammatory cytokines, anti-viral peptides, and regenerative growth factors, using a 27-plex Luminex magnetic bead array and the MagPix® multiplexing system. The following markers were tested: IL-1beta, IL-1ra, IL-2, IL-4, IL-5, IL-6, IL-7, IL-8, IL-9, IL-10, IL-12 (p′70), IL-13, IL-15, IL-17, eotaxin, basic FGF, G-CSF, GM-CSF, IFN-gamma, IP-10, MCP-1 {MCAF}, MIP-1alpha, MIP-1beta, PDGF-BB, RANTES, TNF-alpha, and VEGF. The testing was performed such that all treatments, including each dose of test product and each positive and negative control, were tested in duplicate. The tests were performed once on immune cells from a healthy donor.

Average and standard deviation for each data set, as well as statistical comparison, were calculated using Microsoft Excel. Statistical analysis of the in vitro data was performed by comparison of the repeat measures (triplicates for cell viability and immune cell activation, and duplicates for Luminex cytokine testing) for a specific test condition to relevant controls, For the “tail,” the two-tailed t-test was applied, since a test material may either induce or inhibit a specific assay outcome. For the ““type,” it could be argued that dependent or ‘paired’ analysis would be appropriate, for example, in an assay that uses cells from the same blood donor. However, the independent or ‘unpaired’ test was applied, since each cell culture is unique, positioned in different areas of a microplate with slightly different environmental exposures, and handling through the assay (such as pipetting), although uniform, is not identical. Statistical significance was indicated if p<0.05 and a high level of significance was indicated if p<0.01.

The results are shown in the following order: 1) cell viability testing (including support of cellular energy production); 2) immune cell activation, and cytokine production.

Cell viability testing was performed as a preparatory step to identify the optimal dose range for the in-vitro immune cell testing. Peripheral blood mononuclear cells (PBMC) were used to test serial dilutions of test products for their effects on cell viability in 24 hour cultures. Eleven 4-fold serial dilutions were tested for each product starting at 5 mg/mL. Testing conditions were performed in triplicate and cultures maintained at 37° C., 5% CO₂ for 24 hours. The viability of cells exposed to product dilutions was compared to the viability of untreated cells cultured under the same conditions.

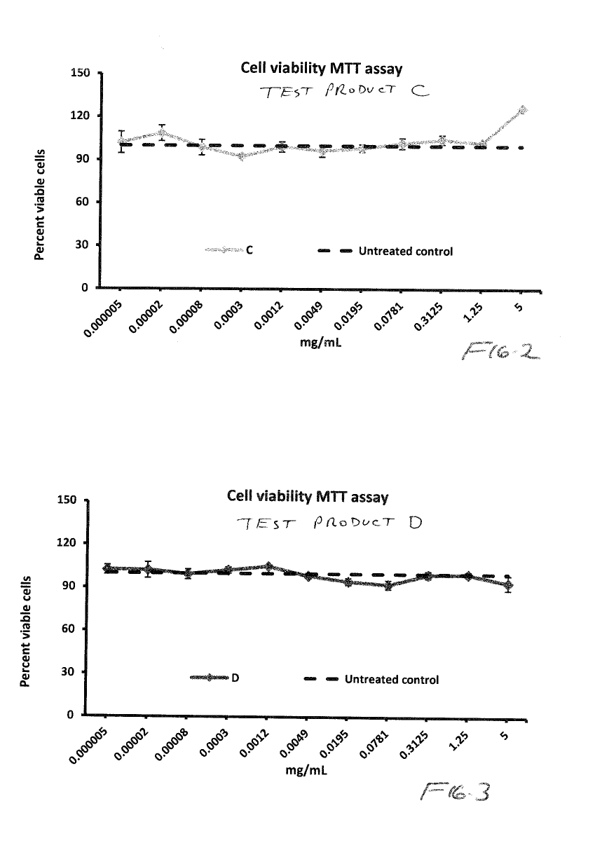

Results are shown in FIGS. 2 and 3 for the water-soluble component derived from the genus Euglena organism as preferably a Euglena gracilis algae (Test Product C) and the sample derived from pure paramylon (TestProduct D). The water-soluble component for Test Product C provided enhancement of cellular viability at higher doses. FIGS. 2 and 3 show cell viability. PBMC were exposed to serial dilutions of the test product for 24 hours, after which time cultures were processed in the colorimetric MTT assay. This assay uses a dye that turns purple as a result of cellular metabolism. Results reflect the sum of the metabolic activity of the cells in each culture. In the graphs of FIGS. 2 and 3, the percent viable cells are shown as the average+/−standard deviation for each triplicate set of cell cultures, compared to untreated cultures.

Immune cell activation was analyzed after the cell viability testing. The phenotypic analysis was performed on the peripheral blood mononuclear cells (PBMC) from one healthy donor. The cells were stained with a combination of monoclonal antibodies to monitor activation, and analyzed by multi-parameter flow cytometry, using an acoustic dual laser Attune® flow cytometer. The analysis involved cellular size and granularity, which is related to activation status, degranulation, and progression of apoptosis. In addition, the analysis included fluorescent markers for CD3, CD56, C069, and CD25. This combination allowed monitoring of changes to monocytes/macrophages, as well as activation of natural killer cells, natural killer T-cells, T lymphocytes, and non-T non-NK lymphocytes.

It is known to those skilled in the art that lymphocytes are a type of round-shaped white blood cell with a round nucleus that occupies the majority of the cell. Three types of cells belong to the lymphocyte class. These are natural killer cells, T lymphocytes and B lymphocytes. Natural killer (NK) cells, also known as large granular lymphocytes, are a type of cytotoxic lymphocyte. They are important for the detection and destruction of virally infected and cancer cells. Natural killer T (NKT) cells are a subset of T lymphocytes that share properties of both T-cells and NK cells. T lymphocytes are a type of lymphocyte that plays a central role in adaptive immunity. Their name is derived from the fact that they mature in the thymus.

B lymphocytes are a type of lymphocyte that collaborate with T lymphocytes and monocytes/macrophages to activate the B cells, which will then differentiate into plasma cells that home to the bone marrow, where they produce antigen-specific immunoglobulins. A subset of the activated B cells will settle in various immune tissue, such as lymph nodes and Peyer's Patches as memory cells, capable of mounting an efficient response if the same antigen is encountered in the future. Monocytes are the largest type of white blood cell. They are present in the blood circulation and become macrophages after leaving the blood and migrating into tissues.

Immune cells in the blood interact with each other and together make up the innate and adaptive arms of the immune system. Innate immunity, also known as cell-mediated immunity is a rapid response usually in reaction to pathogens or transformed cells (virally-infected or cancer) whereas adaptive immunity requires cellular interactions resulting in antigen presentation, immunoglobulin production and the maturation of T lymphocytes into specialized subsets.

The cellular staining for activation, performed using a fluorescently labelled monoclonal antibody to the CD69 and CD25 activation markers, allowed researchers to measure cellular activation by fluorescence intensity. CD69 is a protein expressed on activated white blood cells including T lymphocytes, NK cells, monocytes and PMN cells. CD69 is the earliest inducible cell surface glycoprotein during lymphoid activation resulting in lymphocyte proliferation and cellular signaling. While CD69 plays an important role in immunity through the increase of lymphocyte proliferation and cellular signaling, it has recently been implicated in the immunomodulatory effects leading to the control of inflammation such as noted in the article entitled, “Is CD69 an Effective Brake to Control Inflammatory Diseases,” by Gonzalez-Amaro et al., the disclosure of which is hereby incorporated by reference in its entirety.

CD25 is the alpha chain of the low affinity interleukin-2 (IL-2) receptor (IL-2Ra). CD25 expression is upregulated on activated T lymphocytes, regulatory T-cells and monocytes. Data for immune cell activation in PBMC cultures is presented and are divided into sections according to cell type. Data from one donor is shown. The bar charts in the following figures described below show individual raw data for the comparison of the two samples as the water-soluble component derived from the genus Euglena organism as the preferred dried whole cell and powdered Euglena gracilis algae (Test Product C) and the sample derived from the pure beta-glucan of at least 95% purity paramylon (TestProduct D). Immune cell activation is shown by mean fluorescence intensity as the average±standard deviation for each triplicate set of cell cultures. Statistical significance is indicated on the bar graph (*p<0.05, **p<0.01).

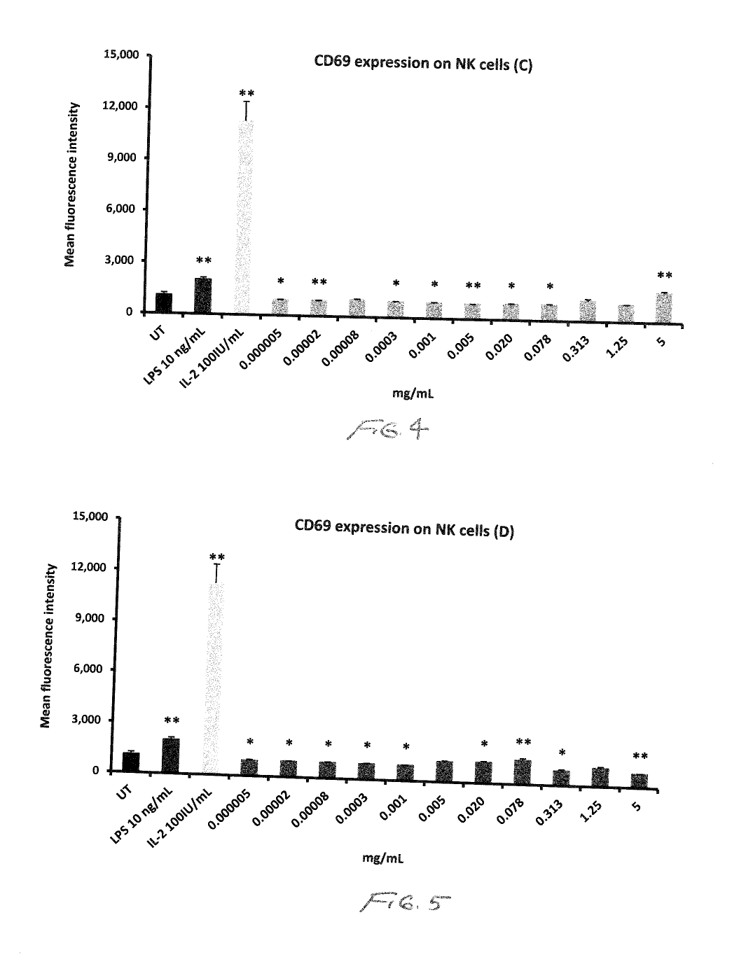

FIG. 4 and FIG. 5 show the bar charts for the Natural Killer (NK) cells CD69 expression of Test Products C and D. Following activation, NK cells express CD69 and therefore an increase in this glycoprotein on the cell surface of NK cells reflects a state of activation. The main downstream effects of CD69 expression by NK cells are associated with increased cytotoxicity. This is explained, for example, in the article entitled, Functional Significance of the Activation-Associated Receptors CD25 and CD19 on Human NK-cells and NK-like T-cells,” by Clausen et al., the disclosure which is hereby incorporated by reference in its entirety. Given this known role of CD69 on NK cells, the increase seen in response to some products suggest that these products support innate immunity, particularly that towards virally-infected or transformed (cancer) cells through the sustained activation and increased cytotoxicity of NK cells.

The water-soluble component derived from the Euglena gracilis algae as Test Product C induced CD69 expression on NK cells, with the potency of Test Product C close to the potency of the positive control LPS. The bar charts of FIGS. 4 and 5 show the CD69 expression for Test Products C and D. These two bar charts show the expression of the CD69 activation marker on NK cells in 24-hour PBMC cultures. The bar charts show the CD69 mean fluorescence intensity as the average+/−standard deviation for each triplicate set of cell cultures. Statistical significance is indicated on the bar chart (*p<0.05, **p<0.01).

There now follows a description of the Natural Killer (NK) cells CD25 expression. CD25 is the alpha chain of the interleukin-2 receptor. It is considered a late marker of activation (in contrast to CD69 which is an early activation marker). Expression of CD25 by NK cells leads to increased proliferation as noted in the article entitled, uIL-2 Triggers Specific Signaling Pathways in Human NKT Cells Leading to Production of Pro- and Anti-Inflammatory Cytokines,” by Bessoles et al., the disclosure which is hereby incorporated by reference in its entirety.

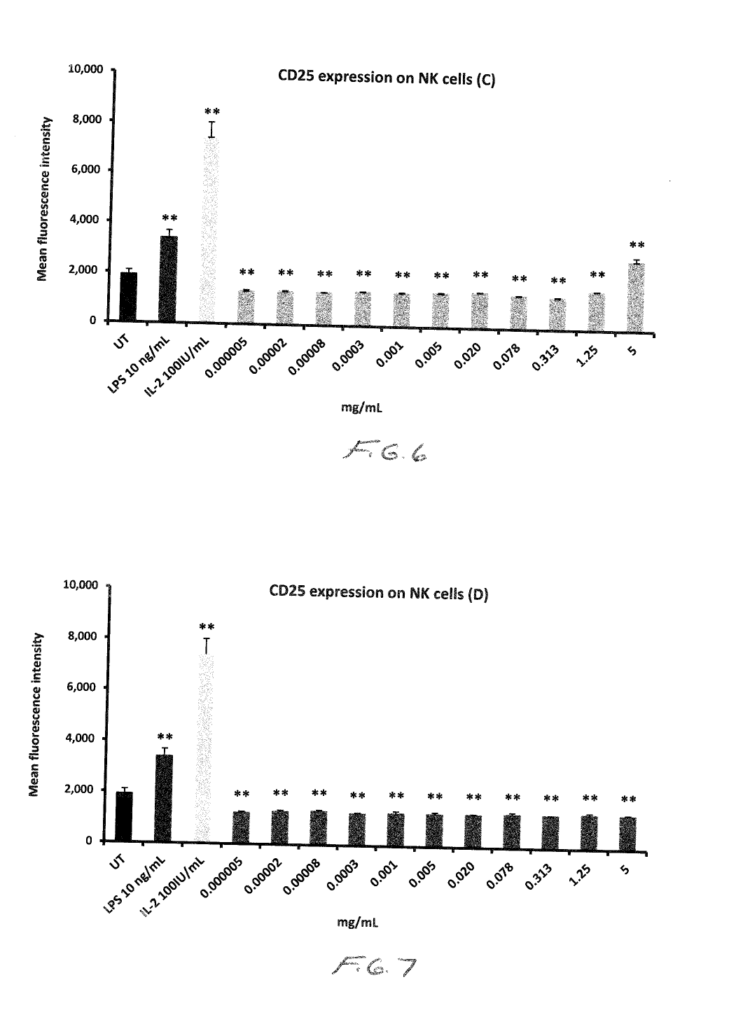

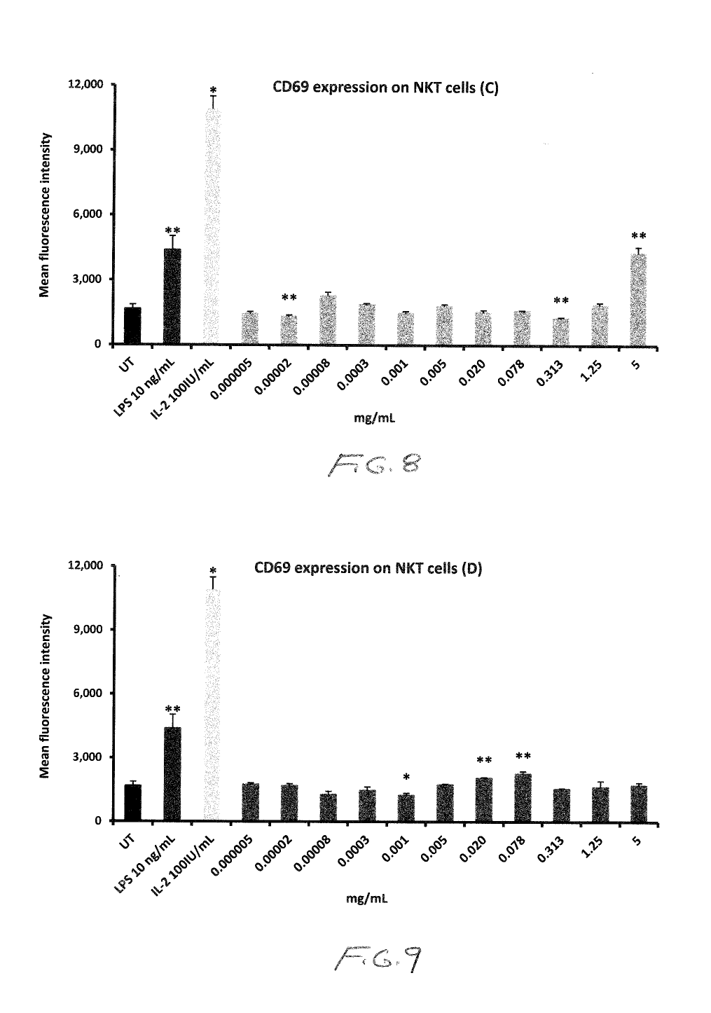

As shown in the bar chart of FIG. 6, the water-soluble component derived from Euglena algae (Test Product C) induced CD25 expression on NK cells. The two bar charts in FIGS. 6 and 7 show expression of the CD25 activation marker on NK cells in 24-hour PBMC cultures. The bar charts show the CD25 mean fluorescence intensity as the average+/−standard deviation for each triplicate set of cell cultures. Statistical significance is indicated on the bar graph (*p<0.05, **p<0.01).

There now follows a description of the Natural Killer T (NKT) cells and CD69 expression. NKT cells share properties with both NK cells and T-cells. Following activation, NKT cells express CD69 and therefore an increase in this glycoprotein on the cell surface of NKT cells reflects a state of activation. NKT cells have regulatory roles in autoimmunity and work alone as well as in concert with CD4+CD25+ regulatory T-cells {Tregs), as noted in the article entitled, “CD4+CD25+ Tregs and NKT Cells: Regulators Regulating Regulators,” by Lacava et al., the disclosure which is hereby incorporated by reference in its entirety.

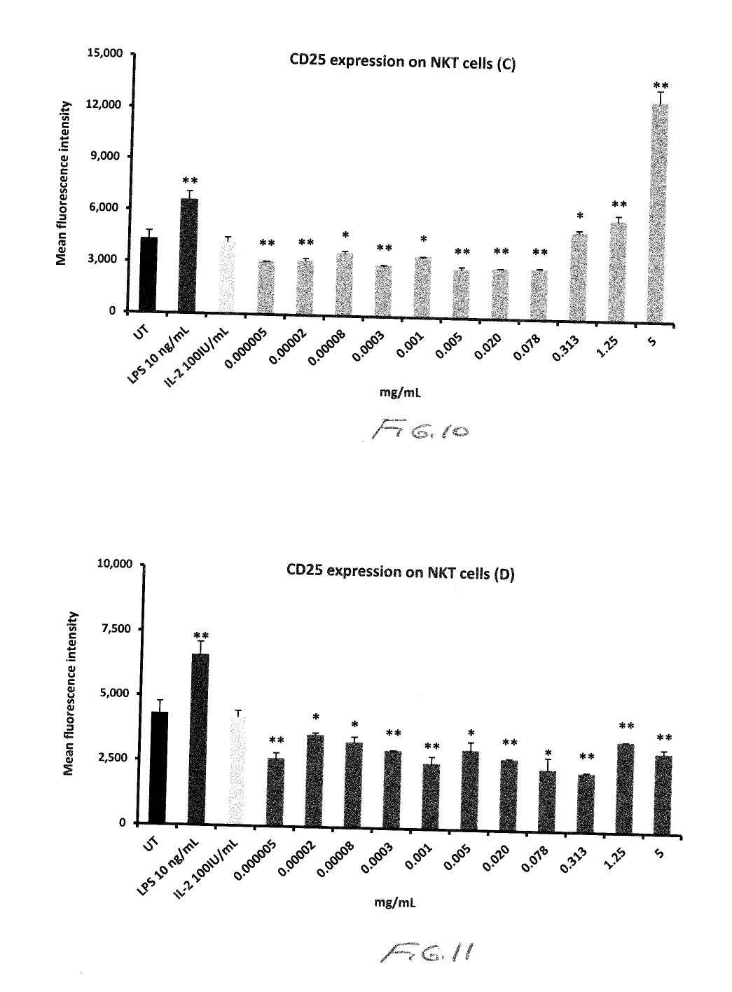

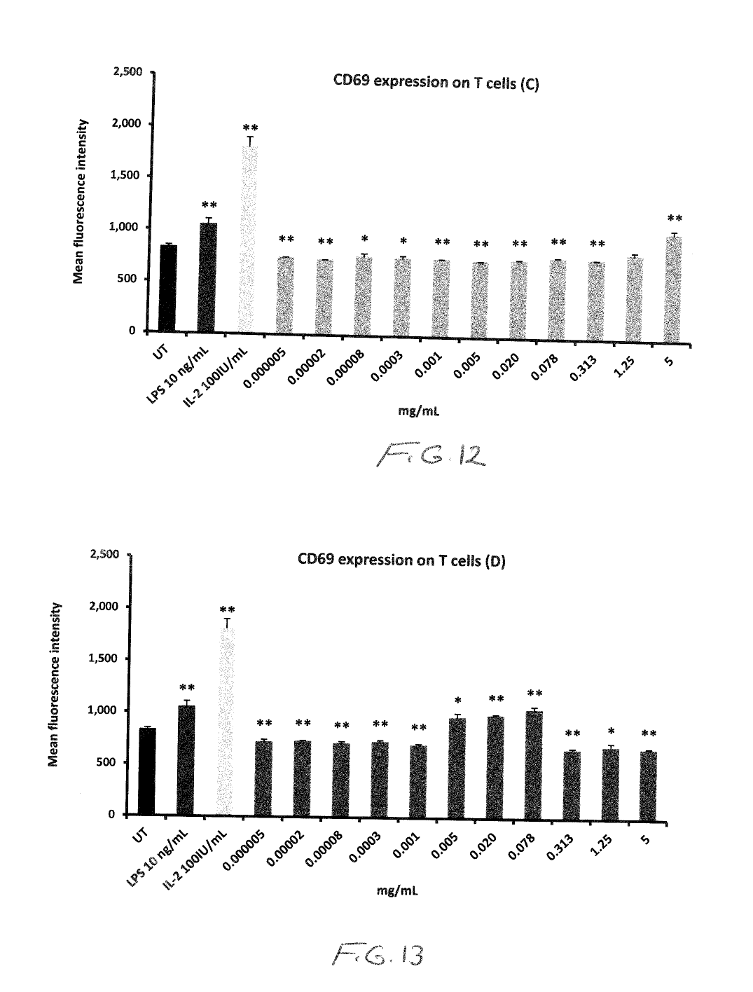

The water-soluble component as derived from the Euglena gracilis algae (Test Product C) strongly induced CD69 expression on NKT cells (FIG. 8), equal to or stronger than the positive control LPS. The bar charts of FIGS. 8 and 9 show expression of the CD69 activation marker on NKT cells in 24-hour PBMC cultures. The bar charts show the CD69 mean fluorescence intensity as the average+/−standard deviation for each triplicate set of cell cultures. Statistical significance is indicated on the bar chart (*p<0.05, **p<0.01).

There now follows an explanation of Natural Killer T (NKT) cells and CD25 expression. NKT cells share properties with both NK cells and T-cells. Up-regulation of CD25 expression on NKT cells reflects a state of activation and results in the production of both pro- and anti-inflammatory cytokines. NKT cells have regulatory roles in autoimmunity and work alone as well as in concert with CD4+CD25+ regulatory T-cells (Tregs). The water-soluble component derived from the Euglena gracilis algae (Test Product C) at higher doses strongly induced CD25 expression on NKT cells, stronger than the positive control LPS (FIG. 10). The bar charts show the expression of the CD25 activation marker on NKT cells in 24-hour PBMC cultures. The bar charts show the CD25 mean fluorescence intensity as the average+/−standard deviation for each triplicate set of cell cultures. Statistical significance is indicated on the bar chart (*p<0.05, **p<0.01).

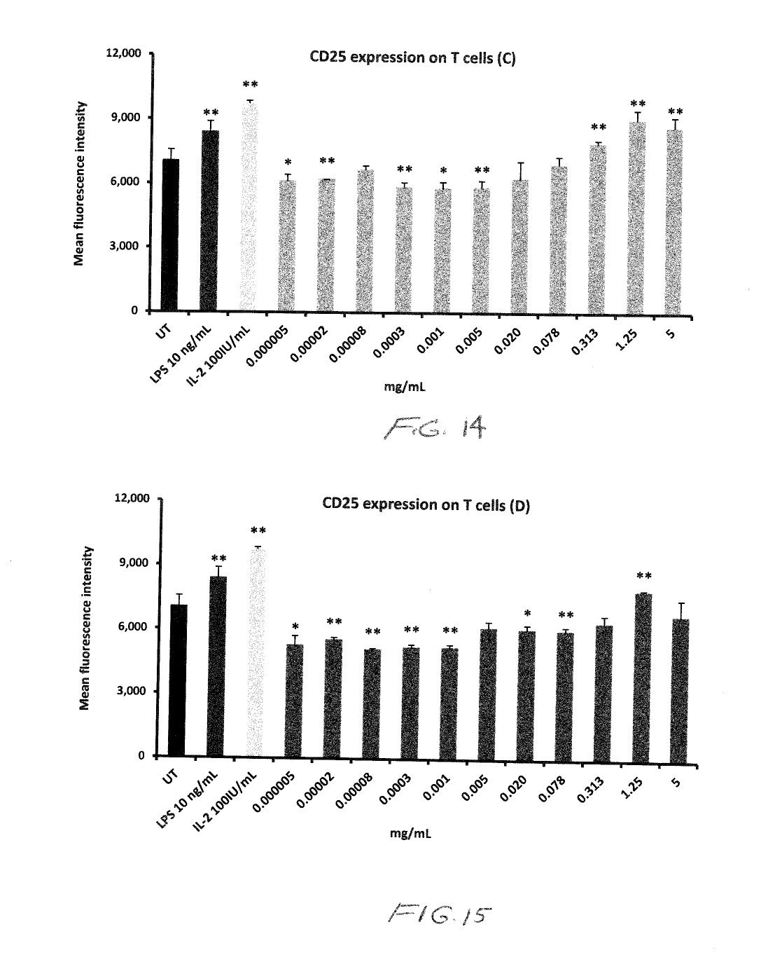

There now follows a description of T-cells and CD69 expression. Activation of T-cells through the T-cell receptor (TCR) results in the up-regulation of a number of proteins including the ‘immediate early activation marker’ CD69. Increased expression of CD69 on T-cells is associated with an increase in T-cell proliferation. Both the water-soluble component derived from the Euglena gracilis algae as Test Product C (FIG. 12) and the insoluble paramylon sample as Test Product D (FIG. 13) only exerted minor effects on CD69 expression on NK cells. These bar charts show expression of the CD69 activation marker on T-cells in 24-hour PBMC cultures. The bar charts show the CD69 mean fluorescence intensity as the average+/−standard deviation for each triplicate set of cell cultures. Statistical significance is indicated on the bar chart (*p<0.05, **p<0.01).

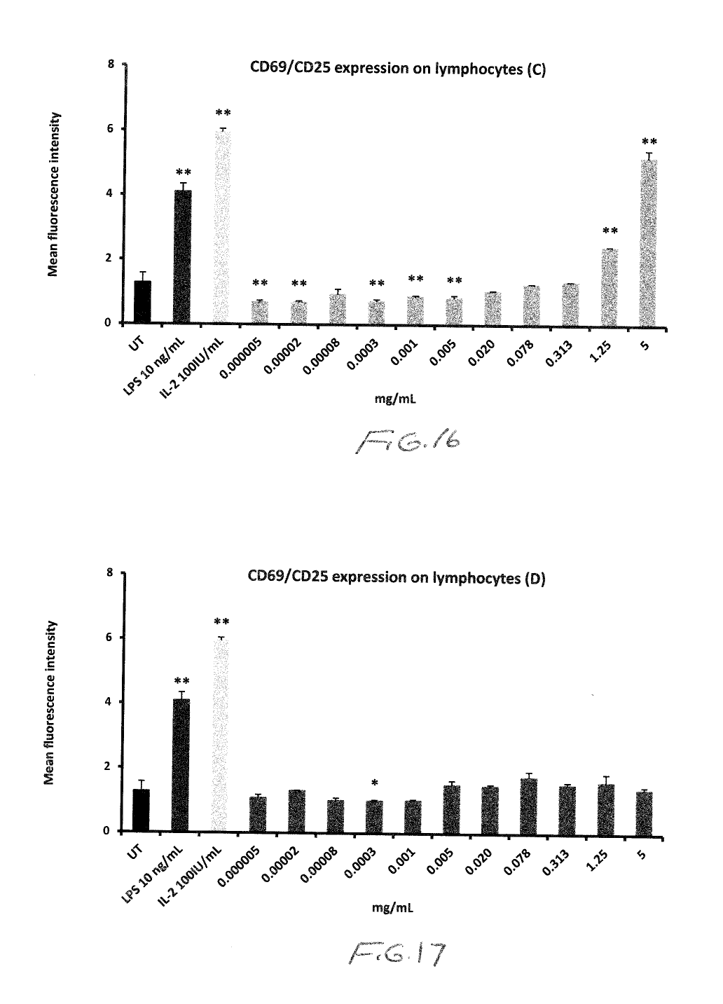

There now follows a description of T-cells and CD25 expression. T-cells expressing CD25 include a special subset of T-cells called regulatory T-cells (Tregs) that play an important role in immunoregulation and tolerance of self-antigens. The water-soluble component derived from the Euglena gracilis algae (Test Product C) at higher doses strongly induced CD25 expression on T-cells, stronger than the positive control LPS (FIG. 14). Both the water-soluble component derived from the Euglena gracilis algae (Test Product C) and the insoluble paramylon sample of Test Product D (FIG. 15) at lower doses showed varying degrees of suppressing CD25 expression on T-cells, suggesting that the T-cells were redirected to other activities than proliferation. These bar charts show expression of the CD25 activation marker on T-cells in 24-hour PBMC cultures. The bar charts show the CD25 mean fluorescence intensity as the average+/−standard deviation for each triplicate set of cell cultures. Statistical significance is indicated on the bar chart (*p<0.05, **p<0.01).

There now follows a description of CD69/CD25 double positive lymphocytes. CD69/CD25 double positive lymphocytes do not constitute a single cell type, but rather this population is made up of a number of different immune cells including subsets of NK, NKT and CD8+ T-cells. Published literature on CD69/CD25 double positive lymphocytes has been minimal, but it has been determined that CD8+ T-cells expressing both activation markers are increased in smokers and is directly correlated to the number of cigarettes consumed, as noted in the article entitled, Differential Activation of Killer Cells in the Circulation and the Lung: A Study of Current Smoking Status and Chronic Obstructive Pulmonary Disease (COPD),” by Wang et al., the disclosure which is hereby incorporated by reference in its entirety.

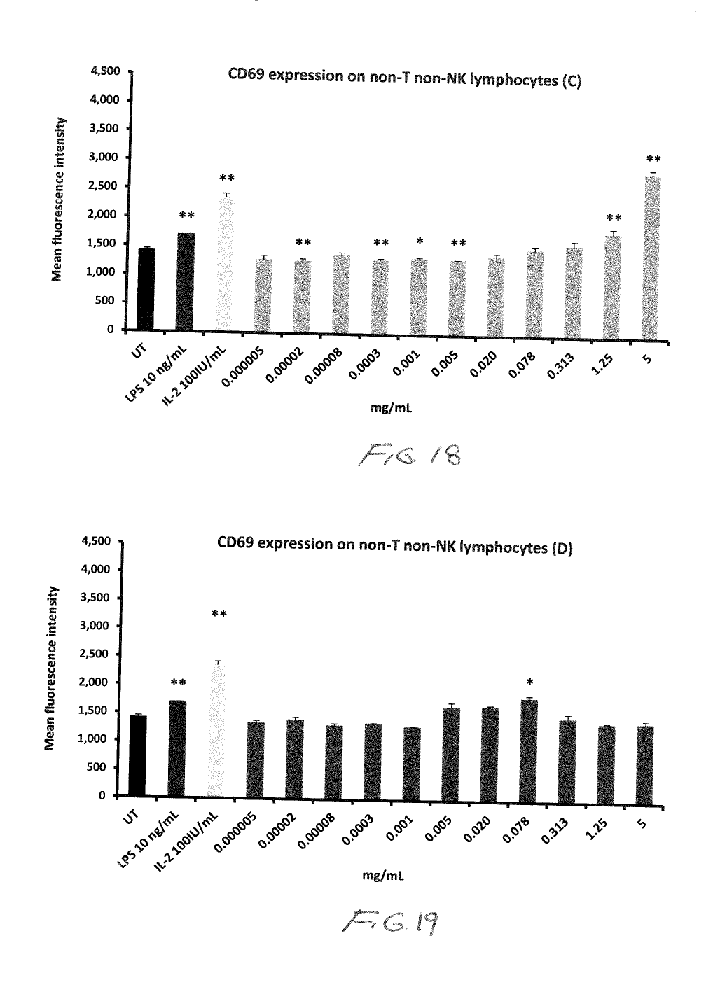

The water-soluble component derived from the Euglena gracilis algae as Test Product C increased the number of CD69/CD25 double-positive lymphocytes in the cell cultures at the highest dose {FIG. 16). The water insoluble paramylon sample as Test Product D (FIG. 17) only exerted minor, if any, effects on the number of CD69/CD25 double-positive lymphocytes. These bar charts show expression of the CD69/CD25 activation marker on lymphocytes in 24-hour PBMC cultures. The bar charts show the CD69/CD25 mean fluorescence intensity as the average+/−standard deviation for each triplicate set of cell cultures. Statistical significance is indicated on the bar chart (*p<0.05, **p<0.01).

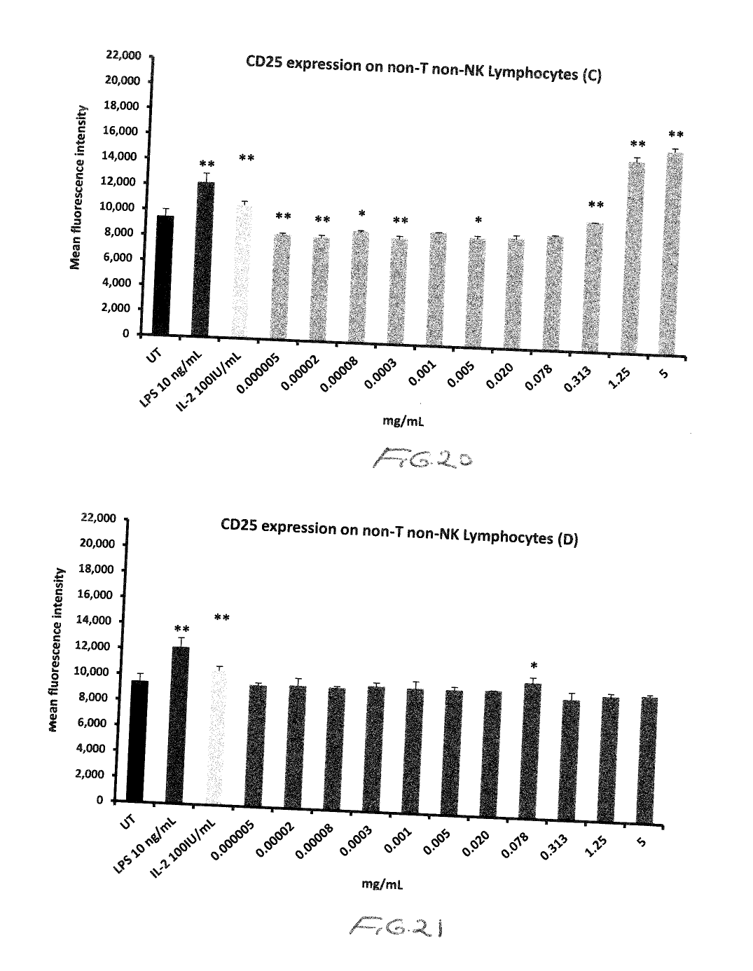

There now follows a description of non-T non-NK lymphocytes and CD69 expression. The remaining portion of the lymphocyte population includes non-T, non-NK cells. This population includes first and foremost the B lymphocytes, and also includes some dendritic cell types, stem cells, and other rare cell types. The water-soluble component derived from the Euglena gracilis algae as Test Product C robustly increased CD69 expression on non-T non-NK cells at the highest dose (FIG. 18). The pure paramylon sample as Test Product D only exerted minor, if any, effects on CD69 expression on non-T non-NK cells (FIG. 19). These bar charts show expression of the CD69 activation marker on non-T and non-NK lymphocytes in 24-hour PBMC cultures. The bar charts show the CD69 mean fluorescence intensity as the average+/−standard deviation for each triplicate set of cell cultures. Statistical significance is indicated on the bar chart (*p<0.05, **p<0.01).

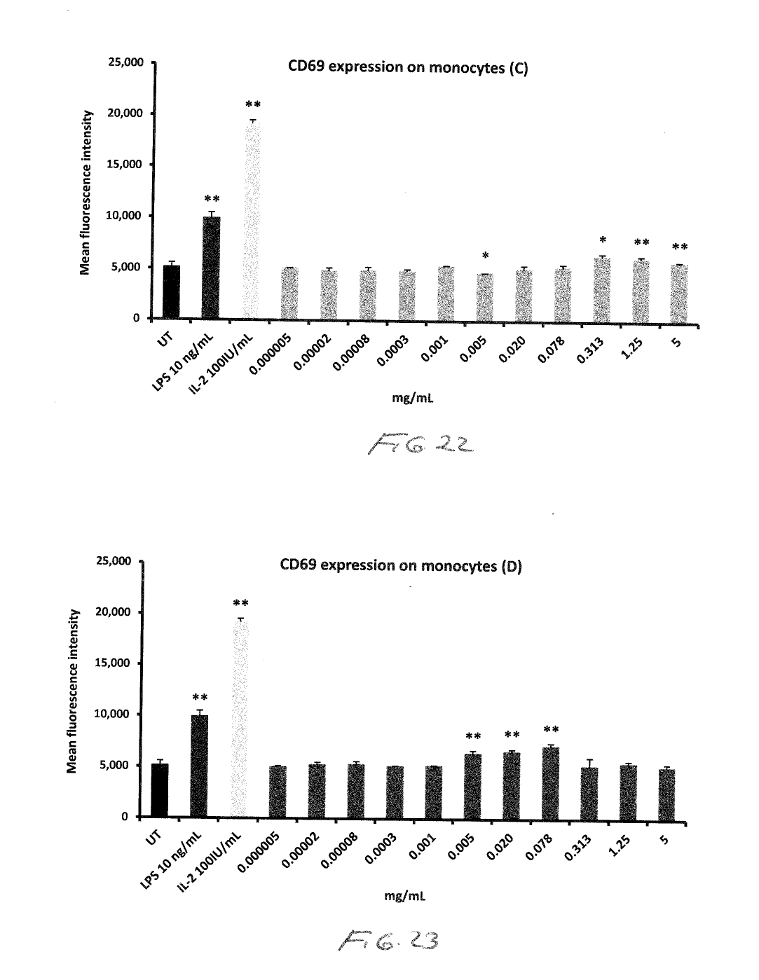

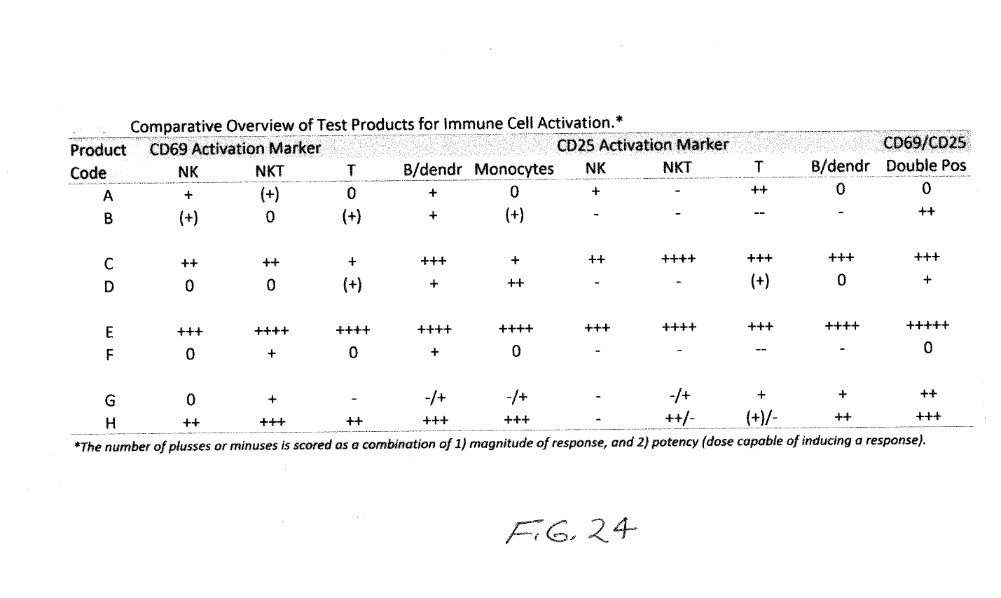

There now follows a description of non-T and non-NK lymphocytes and CD25 expression. Similarly, the water-soluble component derived from the Euglena gracilis algae as Test Product C (FIG. 20) robustly increased CD25 expression on non-T and non-NK cells at higher doses, while the pure paramylon sample as Test Product D (FIG. 21) only exerted minor, if any, effects on CD69 expression on non-T non-NK cells. These bar charts show expression of the CD25 activation marker on non-T and non-NK lymphocytes in 24-hour period cultures. The bar charts show the CD25 mean fluorescence intensity as the average+/−standard deviation for each triplicate set of cell cultures. Statistical significance is indicated on the bar chart (*p<0.05, **p<0.01).

There now follows a description of monocytes and CD69 expression. Monocytes express CD69, and inflammatory responses can be triggered via this cell surface receptor. CD69-mediated inflammatory activation of monocytes triggers unique responses different from CD69-mediated activation of other cell types. The CD69-mediated monocyte response includes production of Prostaglandin E2 alpha, 6-keto-prostaglandin F1 alpha, and leukotriene B4, suggesting the activation of both cyclooxygenase and lipoxygenase pathways after CD69 stimulation. An example is the article entitled, “CD69 is a TGF-/1,25-Dihydroxyvitamin D3 Target Gene in Monocytes,” by Wobke et al., the disclosure which is hereby incorporated by reference in its entirety.

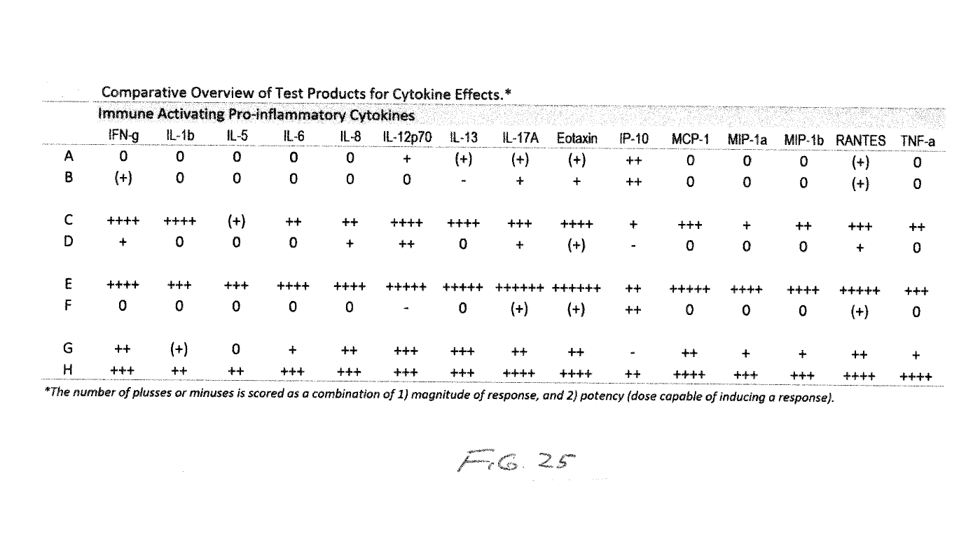

The water-soluble sample derived from the Euglena gracilis algae as Test Product C (FIG. 22) moderately increased CD69 expression on monocytes at some doses. The insoluble paramylon sample as Test Product D (FIG. 23) only exerted minor, if any, effects on CD69 expression on monocytes.

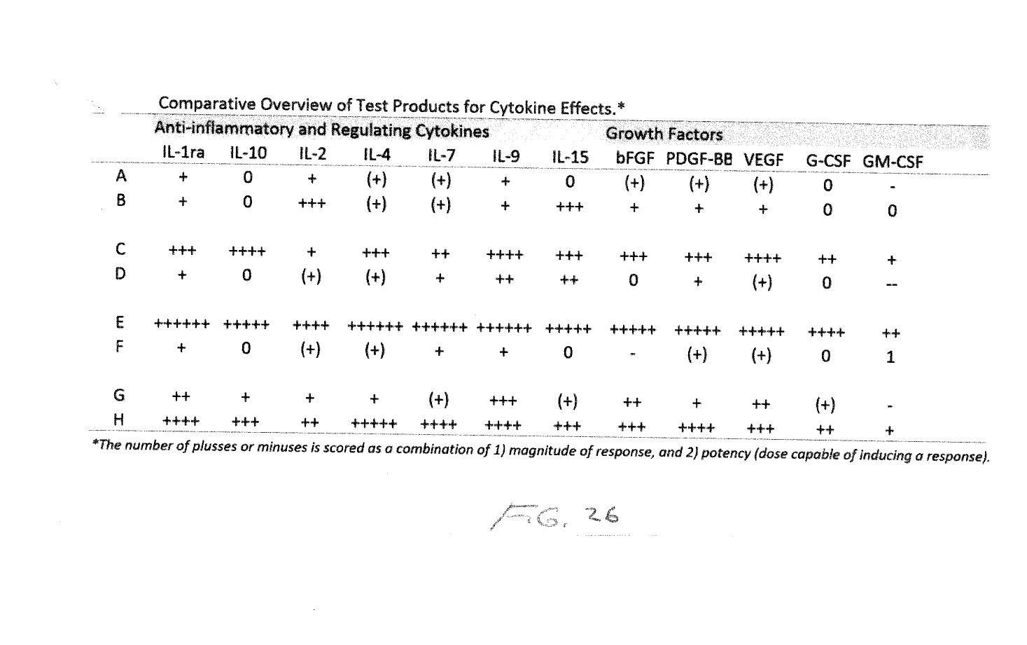

The overview tables shown at FIGS. 24-26 provide a qualitative, comparative summary of the potency of the eight Test Products A to Hand identified above. For simplicity, since in some cases a specific dose range of a Test Product triggered substantial cellular activation and at lower doses a slight inhibition was evident, for the purpose of these Tables a focus was placed on the activation, in some cases, ignoring the bi-phasic responses.

There now occurs a general description for the cytokine testing. In parallel to the testing for immune cell activation, the cell-free culture supernatants from the same PBMC cultures were used for testing of a broad panel of cytokines and chemokines, using a 27-plex Luminex magnetic bead array and the MagPix® multiplexing system. Supernatants from one of each triplicate culture for each test condition were tested in duplicate. Each product was tested at all eleven doses, along with negative {untreated) and positive (LPS) controls.

The 27 cytokines/chemokines and a brief description of their major mode of action is listed below. The markers tested: IL-1beta, IL-1ra, IL-2, IL-4, IL-5, IL-6, IL-7, IL-8, IL-9, IL-10, IL-12 (p70), IL-13, IL-15, IL-17, Eotaxin, basic FGF, G-C SF, GM-CSF, IFN-gamma, IP-10, MCP-1 (MCAF), MIP-1alpha, MIP-1beta, PDGF-BB, RANTES, TNF-alpha, and VEGF.

Pro-Inflammatory Cytokines/Chemokines

| | |
|---|---|
| IFN-y | Interferon gamma. Also called macrophage- |
| | activating factor. Associated with a number of |
| | auto-inflammatory and autoimmune diseases. |
| IL-1 | Interleukin 1 beta. Produced by activated |
| | macrophages as a proprotein which is cleaved by caspase 1. |
| | Important mediator of inflammation. |
| IL-5 | Interleukin 5. Key mediator in eosinophil |
| | activation {allergy). |
| IL-6 | Interleukin 6. Mostly a pro-inflammatory |
| | cytokine. Inhibitor to IL-6 has been developed as drug for |
| | rheumatoid arthritis. |
| IL-8 | Interleukin 8 · Neutrophil chemotactic factor. |
| | Often associated with inflammation. |
| IL-12p70 | Interleukin 12 (protein 70). Produced by activated antigen- |
| | presenting cells. Strong |
| | inducer of interferon gamma. |
| IL-13 | Interleukin 13. Secreted by Th2 helpercells |
| | which are mediators of inflammation. |
| IL-17A | Interleukin 17A. Pro-inflammatory cytokine produced by |
| | activated T-cells. Associated with inflammatory diseases |
| | (rheumatoid arthritis, |
| | psoriasis, multiple sclerosis. |
| Eotaxin | Eosinophil chemotactic protein (CCLll). |
| | Implicated in allergic responses. |
| IP-10 | Interferon gamma-induced protein 10 (CXCLlO). |
| | Associated with inflammation. |
| MCP-1 | Monocyte chemotactic protein-1 (CCL2). Recruits cells to |
| | sites of inflammation produced by |
| | injury or infection. |
| MIP-lcx | Macrophage Inflammatory Protein 1 alpha (CCL3). Produced |
| | by macrophages stimulated by bacterial endotoxins. Crucial |
| | for immune responses to infection and inflammation. |
| | Activates neutrophils, induces pro-inflammatory |
| | cytokines. |
| MIP-1 | Macrophage Inflammatory Protein 1 beta (CCL4). |
| | Pro-inflammatory. See MIP-1a description above. |
| RANTES | Regulated on Activation, Normal T cell Expressed and |
| | Secreted {CCLS). Chemotactic for T-cells, eosinophils and |
| | basophils. Plays active role in recruiting leukocytes into |
| | inflammatory sites. |
| TNF-a. | Tumor necrosis factor alpha. Adipokine involved in systemic |
| | inflammation. Produced mainly by activated macrophages. |
| | Stimulates acute phase |
| | reaction. |

Anti-Inflammatory Cytokines/Chemokines

| | |
|---|---|
| IL-lra | Interleukin-1 receptor antagonist. Natural inhibitor |
| | of the pro-inflammatory effects of IL-1. |
| IL-10 | Interleukin 10. Anti-inflammatory cytokine but |
| | requires activation of cells to induce. |
| IL-2 | Interleukin 2. Necessary for the growth, proliferation and |
| | differentiation of T-cells. Part of the body's natural response |
| | to microbial infection. Also important for discriminating |
| | between “non-self” and “self.” |
| IL-4 | Interleukin 4. Induces naive T-cells (TO) to become |
| | Th2. Overproduction associated with allergies. |
| IL-7 | Interleukin 7. Hematopoietic growth factor secreted by |
| | stromal cells in the bone marrow and thymus. |
| | Stimulates differentiation of hematopoietic stem cells |
| | into lymphoid progenitor cells. Stimulates proliferation of |
| | B cells, T-cells and NK cells. |
| IL-9 | Interleukin 9. Cytokine produced by T-cells - particularly |
| | CD4+ helper cells. Identified as a |
| | candidate gene for asthma. |
| IL-15 | Interleukin 15. Secreted by mononuclear phagocytes |
| | following infection by virus(es). Induces proliferation of NK cells. |

Growth Factors

| | |
|---|---|
| bFGF | Basic Fibroblast Growth Factor. Important in |
| | angiogenesis and wound healing. |
| POGF-BB | Platelet-Derived Growth Factor subunit Beta. Involved in |
| | angiogenesis and is a potent mitogenfor cells of mesenchymal |
| | origin. Important for wound healing. |
| VEGF | Vascular Endothelial Growth Factor. Stimulates |
| | vasculogenesis and angiogenesis. Serum concentration of |
| | VEGF is high in bronchial asthma and diabetes mellitus. |
| G-CSF | Granulocyte Colony-Stimulating Factor. Promotes |
| | proliferation of neutrophils. |
| GM-CSF | Granulocyte-Macrophage Colony-Stimulating Factor. |
| | Secreted by macrophages, T-cells, mast cells, NK cells, |
| | endothelial cells and fibroblasts. Leukocyte growth factor that |
| | stimulates stem cells to produce granulocytes and monocytes. |
| | It is part of the immune/inflammatory cascade by leading to |
| | the activation of monocytes. |

The three overview Tables at FIGS. 24-26 provide a qualitative, comparative summary of the potency of these eight Test Samples A-H described above. For simplicity, since in some cases the higher dose range of a Test Product triggered substantial cytokine release, and at lower doses a slight inhibition was seen, these Tables focus on the immune activation, thus in some cases ignoring bi-phasic responses. It should be understood that the anti-inflammatory effect seen at lower doses of several products may also be of importance.

These results in these three Tables show that the water-soluble component derived from the genus Euglena organism (Test Product C) contains a potent water-soluble antigen not related to the insoluble and high molecular weight paramylon also found in the genus Euglena organism as in Test Product D. Because the pure paramylon sample preparation corresponding to Test Product D after the 0.2 micron filtration contained very little, if any, paramylon or Euglena water-soluble component, it showed essentially no activity in the in vitro screen. The water-soluble component (Test Product C), on the other hand, showed the very broad and potent immune cell activation that caused extensive release of immune cross-talk cytokines generated by the immune cells coming into direct contact with the water-soluble component and increased the absolute populations of many immune cell types.

What is claimed is:

1. A method for promoting the immune health of an animal or human by administering a composition that contains an effective amount of at least one component derived from the genus Euglena, wherein the at least one component is capable of stimulating immune system activity in the absence of beta-glucan.

2. The method according to claim 1 wherein the Euglena is fermented heterotrophically, mixotrophically and/or phototrophically, or any combination thereof.

3. The method according to claim 1 wherein the mixture is derived from Euglena gracilis.

4. The method according to claim 1 wherein the composition is delivered as a human dietary supplement in a suspension, capsule or tablet form.

5. The method according to claim 1 wherein the composition is delivered as an ingredient in food.

6. The method according to claim 1 wherein the composition is administered in combination with other innate or adaptive immune modulating compounds.

7. The method according to claim 6 wherein the other immune modulating compounds are selected from the following group: a probiotic, an inactivated bacteria, a beta-1,3; 1,6-glucan, a beta-1,3-glucan, an algae extract or a carotenoid.

8. The method according to claim 7 wherein the probiotic is a Bacillus or Lactobacillus.

9. The method according to claim 7 wherein the inactivated bacteria is a Bacillus or Lactobacillus.

10. The method according to claim 7 wherein the beta-glucan is derived from a yeast, mushroom or Euglena.

11. The method according to claim 7 wherein the algae extract is derived from an Arthrospira or Haematococcus.

12. The method according to claim 7 wherein the carotenoid is astaxanthin, lutein, zeaxanthin or combinations thereof.

13. A composition for promoting the immune health of an animal or human comprising at least one water-soluble component derived from the genus Euglena, wherein the water-soluble component is capable of stimulating immune system activity in the absence of beta-glucan.

14. The composition according to claim 13 wherein the water-soluble component is derived from a heterotrophically, mixotrophically and/or phototrophically grown Euglena organism.

15. The composition according to claim 13 wherein the water-soluble component is derived from Euglena gracilis.

16. The composition according to claim 13 wherein the water-soluble component is delivered as a human dietary supplement in a suspension, capsule or tablet form.

17. The composition according to claim 13 wherein the water-soluble component is delivered as an ingredient in food.

18. The composition according to claim 13 further comprising other innate or adaptive immune modulating compounds.

19. The composition according to claim 18 wherein the other immune modulating compounds are selected from the following group: a probiotic, an inactivated bacteria, a beta-1,3; 1,6-glucan, a beta-1,3-glucan, an algae extract or a carotenoid.

20. The composition according to claim 19 wherein the probiotic is a Bacillus or Lactobacillus.

21. The composition according to claim 19 wherein the inactivated bacteria is a Bacillus or Lactobacillus.

22. The composition according to claim 19 wherein the beta-glucan is derived from a yeast, mushroom or Euglena.

23. The composition according to claim 19 wherein the algae extract is derived from an Arthrospira or Haematococcus.

24. The composition according to claim 19 wherein the carotenoid is astaxanthin, lutein, zeaxanthin or combinations thereof.

25. A method of improving weight gain, feed intake, feed efficiency and/or lower mortality in an animal by administering at least one water-soluble component derived from the genus Euglena that is capable of stimulating immune system activity in the absence of beta-glucan.
